(19) United States
(12) Patent Application Publication
(10) Pub. No.: US 2023/0405065 A1
(43) Pub. Date: Dec. 21, 2023

(54) MODIFIED HTERT PROMOTER FOR REGULATING CANCER CELL-SPECIFIC GENE EXPRESSION AND ANTI-TUMOR ADENOVIRUS CONTAINING SAME

(71) Applicant: CURIGIN CO., LTD., Seoul (KR)

(72) Inventors: Seong Hoon PARK, Jeollabuk-do (KR); Jung Ki YOO, Yangju-si (KR); Ki Hwan UM, Ansan-si (KR)

(73) Assignee: CURIGIN CO., LTD., Seoul (KR)

(21) Appl. No.: 18/037,487

(22) PCT Filed: Oct. 14, 2021
(86) PCT No.: PCT/KR2021/014180

§ 371 Date: May 17, 2023

(30) Foreign Application Priority Data
Nov. 19, 2020 (KR) 10-2020-0155505

(51) Int. Cl. A61K 35/761; C12N 7/00; C12N 15/86; A61P 35/00

(52) U.S. Cl. CPC A61K 35/761; C12N 7/00; C12N 15/86; A61P 35/00

(57) ABSTRACT

The present invention relates to a modified hTERT promoter for regulating cancer cell-specific gene expression and an oncolytic adenovirus including the same. The modified hTERT promoter of the present invention increases the cancer-specific transcriptional activity of a target gene compared to the case where a wild-type hTERT promoter is used, and when the modified hTERT promoter is used for an oncolytic adenovirus, the tumor-killing effect is excellent compared to the case where an adenovirus is prepared using the wild-type hTERT promoter.

### TECHNICAL FIELD

The present invention relates to a modified hTERT promoter for regulating cancer cell-specific gene expression and an oncolytic adenovirus including the same.

### BACKGROUND ART

A human telomere reverse transcriptase (hTERT) is a component of telomerase, which is an enzyme involved in maintaining a constant telomere length during chromosome replication, and is known to be involved in cell aging, cancer, and cell immortality. The activity of telomerase is observed in germ cells of the ovary and the testis and lymphocytes in humans, but is not observed in other normal somatic cells. Accordingly, in the normal somatic cells, after a limited number of cell divisions, the telomere length is reduced to a certain length or less, so that the cells cannot divide any more and die. On the other hand, the telomerase activity is increased in benign tumor cells in a pre-cancer stage and cancer cells. Since the increased activity of telomerase in ovarian cancer was first reported, the increased activity of telomerase has been observed in almost all human cancers, including blood cancer, stomach cancer, lung cancer, liver cancer, colon cancer, brain cancer, prostate cancer, head and neck cancer, breast cancer, and the like. It was found that the expression of hTERT, which plays an essential role in the function of telomerase, is related to the activity of telomerase, and the expression of telomerase is regulated according to the activity of the hTERT promoter, that is, the amount of mRNA of the hTERT. The minimum size of the promoter for regulating the hTERT activity is 181 bp, and a wild-type hTERT promoter includes two c-Myc binding sites and five Sp 1 binding sites. It was reported that the binding of the binding sites with c-Myc and Sp 1, which were oncoproteins expressed much higher in cancer cells than in normal cells, activated the hTERT promoter, and it was observed that the activity of the hTERT promoter was increased by the overexpression of c-Myc. However, there were limitations in achieving sufficient cancer cell specificity with only the wild-type hTERT promoter.

Meanwhile, gene therapy is a technology for treating a disease by directly introducing a gene having a function capable of removing the cause of a disease into a patient's body. Typical examples include a method of restoring a lost function by inserting a wild-type gene into a gene that causes a disease due to loss of the function due to mutation, a method of delivering and expressing a gene that may selectively kill cells that cause a disease, such as cancer, only in specific target cells, and the like. In the latter case, many attempts have been made to develop a promoter that specifically overexpresses a target gene for gene therapy only in cancer cells. Promoters that have been developed in the art so far include baculoviral IAP repeat containing 5 (BIRC5, survivin) and telomerase reverse transcriptase (TERT). It was confirmed that the two genes were expressed in a cancer-specific manner, but the TERT was reported to have strong cancer-specificity, but weak transcriptional activity. Therefore, to compensate for this, a modified promoter was developed by inserting a binding nucleotide sequence of hypoxiainducible factor 1 (HIF-1α), which is a transcription factor that is activated in anoxia as a cancer cell-specific environment, into the TERT promoter, and various attempts have been made to improve the promoter performance. However, these methods also had limitations in inducing cancer cell specificity and strong transcriptional activity of the TERT promoter.

### DISCLOSURE

### Technical Problem

An object of the present invention is to provide an hTERT promoter variant having high cancer cell specificity and strong transcriptional activity, and an adenovirus having strong tumor killing ability into which the promoter is introduced.

### Technical Solution

Therefore, the present invention provides a promoter in which a 228th nucleotide sequence of an hTERT promoter is substituted from C to T; and/or a 250th nucleotide sequence is substituted from C to T.

The present invention provides a composition for overexpressing a target gene in a cancer cell-specific manner including a cancer cell-specific promoter in which a 228th nucleotide sequence of an hTERT promoter is substituted from C to T; and/or a 250th nucleotide sequence is substituted from C to T; and
a target gene operably linked to the promoter.

The present invention provides an oncolytic adenovirus including a bladder cancer cell-specific promoter in which a 228th nucleotide sequence of an hTERT promoter is substituted from C to T; and/or a 250th nucleotide sequence is substituted from C to T.

### Advantageous Effects

According to the present invention, the modified hTERT promoter increases the cancer-specific transcriptional activity of a target gene compared to the case where a wild-type hTERT promoter is used, and when the modified hTERT promoter is used for an oncolytic adenovirus, the tumor-killing effect is excellent compared to the case where an adenovirus is prepared using the wild-type hTERT promoter.

### DESCRIPTION OF DRAWINGS

FIG. 1 is a diagram illustrating an adenovirus into which an hTERT promoter of the present invention is introduced.

FIG. 2 is a diagram illustrating productivity of the adenovirus into which the hTERT promoter of the present invention is introduced.

FIG. 3A is a schematic diagram of analysis of viral genome copies produced by qPCR of the adenovirus into which the hTERT promoter of the present invention is introduced.

FIG. 3B is a diagram illustrating E4 productivity of the adenovirus into which the hTERT promoter of the present invention is introduced.

FIG. 4 is a diagram illustrating tumor killing ability of the adenovirus into which the hTERT promoter of the present invention is introduced.

FIG. 5 is a diagram of confirming tumor killing ability of the adenovirus into which the hTERT promoter of the present invention is introduced.

FIG. 6 is a diagram of confirming E1A expression ability of the adenovirus into which the hTERT promoter of the present invention is introduced.

### MODES OF THE INVENTION

Hereinafter, embodiments of the present invention will be described in detail. However, the following embodiments are presented as examples of the present invention, and the present invention is not limited thereby, and various modifications and applications of the present invention can be made within the description of claims to be described below and equivalents interpreted therefrom.

Unless otherwise indicated, a nucleic acid is recorded in a 5′—>3′ direction from left to right. A numerical range enumerated within the specification is inclusive of numbers defining the range and includes each integer or any non-integer fraction within a defined range.

Unless defined otherwise, all technical and scientific terms used herein have the same meaning as commonly understood by those skilled in the art to which the present invention pertains. Although any methods and materials similar or equivalent to those described herein can be used in the practice for testing the present invention, the preferred materials and methods are described herein.

An aspect of the present invention relates to a promoter in which a 228th nucleotide sequence of an hTERT promoter is substituted from C to T; and/or a 250th nucleotide sequence is substituted from C to T.

In an embodiment, the promoter may further include a substitution selected from the group consisting of a substitution from T to C in a 129th nucleotide sequence; a substitution from C to T in a 235th nucleotide sequence; a substitution from C to T of a 236th nucleotide sequence; a substitution from C to T of a 249th nucleotide sequence; and/or a substitution from A to C of a 317th nucleotide sequence of the hTERT promoter, and further include a deletion of a 245th nucleotide sequence.

In an embodiment, the promoter may overexpress the target gene in a cancer cell-specific manner.

In an embodiment, the cancer cells may be cancer cells selected from the group consisting of bladder cancer, urothelial cancer, liver cancer, colorectal cancer, skin cancer, glioblastoma, melanoma, sarcoma, medulloblastoma, glioma, astrocytoma, adenoid glioma, anaplastic oligoastrocytoma, head and neck cancer, brain cancer, thyroid cancer, papillary thyroid carcinoma, adrenocortical carcinoma, ovarian cancer, uterine clear cell carcinoma, cervical squamous cell carcinoma, mantle cell lymphoma, fibrosarcoma, myxoid liposarcoma, meningioma and renal cell carcinoma, preferably bladder cancer, but is not limited thereto.

In an embodiment, the promoter may be a promoter represented by SEQ ID NO: 2.

In an embodiment, the promoter may be a promoter represented by SEQ ID NO: 3.

In an embodiment, the promoter may be a promoter represented by SEQ ID NO: 4.

Another aspect of the present invention relates to a composition for overexpressing a target gene in a cancer cell-specific manner including a cancer cell-specific promoter in which a 228th nucleotide sequence of an hTERT promoter is substituted from C to T; and/or a 250th nucleotide sequence is substituted from C to T; and a target gene operably linked to the promoter.

In an embodiment, the target gene may be at least one gene selected from the group consisting of a cancer cell death gene, a fluorescent protein gene, a cancer cell suppressor gene, an antigenic gene, a cytotoxic gene, a cell proliferation inhibitory gene, a cytokine gene, a pro-apoptotic gene, and an anti-angiogenic gene.

In an embodiment, the cancer cell death gene may be a BCL-2 family pro-apoptotic gene or a death receptor/ligand gene.

In an embodiment, the fluorescent protein gene may be at least one selected from the group consisting of luciferase, an enhanced green fluorescent protein (EGFP), a green fluorescent protein (GFP), a yellow fluorescent protein (YFP), a red fluorescent protein (RFP), and a cyan fluorescent protein (CFP).

In an embodiment, the cancer cell suppressor gene may be at least one selected from the group consisting of a p53 gene, an APC gene, a DPC-4/Smad4 gene, a BRCA-1 gene, a BRCA-2 gene, a WT-1 gene, an MMAC-1 gene, an MMSC-2 gene, an NF-1 gene, an MTS1 gene, a CDK4 gene, an NF-1 gene, an NF-2 gene, and a VHL gene.

In the present invention, the term “operably linked” means a functional linkage between a gene expression regulatory sequence (e.g., a promoter, a signal sequence, or an array of transcriptional regulatory factor binding sites) and another gene sequence, and accordingly, the regulatory sequence regulates the transcription and/or translation of the another gene sequence.

Another aspect of the present invention relates to an oncolytic adenovirus including a bladder cancer cell-specific promoter in which a 228th nucleotide sequence of a TERT promoter is substituted from C to T; and/or a 250th nucleotide sequence is substituted from C to T.

In an embodiment of the present invention, the adenovirus may have increased tumor killing ability compared to an adenovirus including a wild-type hTERT promoter.

In an embodiment, an endogenous gene of the adenovirus has a structure of 5′ITR; in which the C1 includes E1A, E1B or E1A-E1B; the C2 includes E2B-L1-L2-L3-E2A-L4; the C3 does not include E3 or includes E3; the C4 includes L5; and the C5 may not include E4 or may include E4, and may include a nucleotide sequence represented by SEQ ID NO: 58.

In an embodiment, the adenovirus may be partially deleted in the E3 region.

In an embodiment, the adenovirus may include an expression cassette, and the expression cassette may be located at the C3 region of the endogenous gene of the adenovirus.

In an embodiment, the hTERT promoter may be operably linked to E1A and E1B of the endogenous gene of the adenovirus.

In an embodiment, an IRES sequence may be further included between E1A and E1B of the endogenous gene of the adenovirus.

In an embodiment, the adenovirus may have increased E1A expression compared to the adenovirus including the wild-type hTERT promoter.

Another aspect of the present invention relates to a method for treating cancer, including administering a pharmaceutically effective dose of the oncolytic adenovirus to a subject.

The treatment method of the present invention includes administering a therapeutically effective dose of the oncolytic adenovirus to a subject. It is preferred that a specific therapeutically effective dose for a specific subject is differently applied depending on various factors including the kind and degree of a response to be achieved, a specific composition including whether other agents are used in some cases, the age, body weight, general health conditions, sex, and diet of a subject, an administration time, an administration route, a secretion rate of the composition, a duration of treatment, and a drug used in combination or simultaneously with the specific composition, and similar factors well known in the medical field. A daily dose may be 0.0001 to 100 mg/kg, preferably 0.01 to 100 mg/kg, based on the amount of the pharmaceutical composition of the present invention, and may be administered 1 to 6 times a day. However, it is obvious to those skilled in the art that the dosage or dose of each active ingredient should not include too high of the content of each active ingredient to the extent that may cause side effects. Therefore, the effective dose of the composition suitable for the purpose of the present invention is preferably determined in consideration of the aforementioned matters.

The subject is applicable to any mammal, and the mammal includes not only humans and primates, but also livestock such as cattle, pigs, sheep, horses, dogs, and cats.

The pharmaceutical composition of the present invention may be administered to mammals such as mice, rats, livestock, and humans through various routes. All methods of administration may be expected, and for example, the pharmaceutical composition may be administered by oral, rectal or intravenous, intramuscular, subcutaneous, intrauterine dural or intracerebroventricular injection.

Hereinafter, the present invention will be described in more detail with reference to the following Examples. However, the following Examples are only intended to embody the contents of the present invention, and the present invention is not limited thereto.

### [Example 1] Preparation of Adenovirus Introduced with hTERT Promoter of the Present Invention

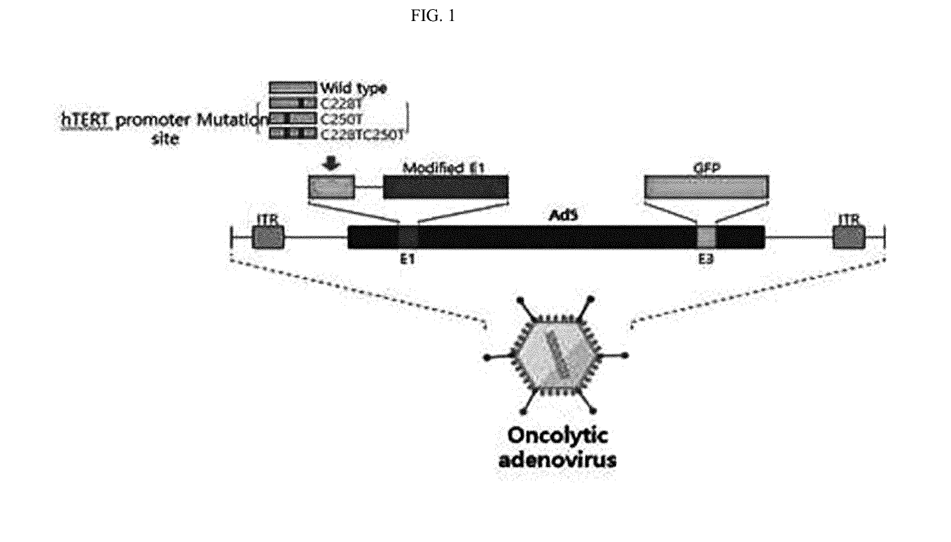

As illustrated in FIG. 1, an adenovirus introduced with an hTERT promoter of the present invention was prepared.

More specifically, a viral vector was prepared using four shuttle vectors pAd1127, pAd1128, pAd1129, and pAd1130 including a genome of the adenovirus. Among them, the pAd1127 vector included early genes E1A and E1B of the adenovirus, and attempted cancer-specific replication by substituting an E1 promoter for regulating E1 expression with a cancer-specific hTERT promoter in a wild-type adenovirus. To prepare a mutant virus in which C at positions −124 and −146 of the hTERT promoter was substituted with T, a point mutation was induced at a specific position of the hTERT promoter in pAd1127. A cosmid including a full genome was prepared by ligating the pAd1127 vector having the mutant type with the pAd1128, pAd1129, and pAd1130 vectors containing the late gene of the adenovirus, and then packed in λ phage to be infected to E. coli and transformed. Thereafter, clones including the genome of the adenovirus were confirmed by preparing a plasmid in E. coli and then analyzing a pattern using a restriction enzyme, and viruses having point mutation were accurately confirmed through sequencing.

### [Example 2] Confirmation of Productivity or Infectivity of Adenovirus Introduced with hTERT Promoter of the Present Invention

HEK-293 cells were infected in 0.1 MOI of adenoviruses introduced with hTERT promoters hTERT C228T, C250T, and C228T/C250T of the present invention, respectively, and then after 48 hours, the infection was observed.

More specifically, 2×10⁶ cells of HEK-293 cells were inoculated in a 6-well plate, and then the cells included in each well were infected in 0.1 MOI. After 48 hours, the cells included in each well and the cells in a virus culture medium were lyzed to recover the produced virus. Thereafter, in order to quantify the produced infectious virus, 1×10⁶ cells of HEK-293 cells were inoculated in a 12-well plate once again, and then serially diluted (serial dilution; 10⁻¹, 10⁻², 10⁻³, 10⁻⁴, 10⁻⁵, and 10⁻⁶) and 100 μl of the cells were added into each well and reinfected. After 48 hours, the cells were immobilized with 100% methanol and treated with a mouse anti-hexon antibody (1:1000) for 1 hour. After washing each well, the cells were treated with a rabbit-anti-mouse-HRP antibody (1:500) for 1 hour. Each well was washed once again, and the productivity of the virus was measured by counting a portion colored by a DAB solution.

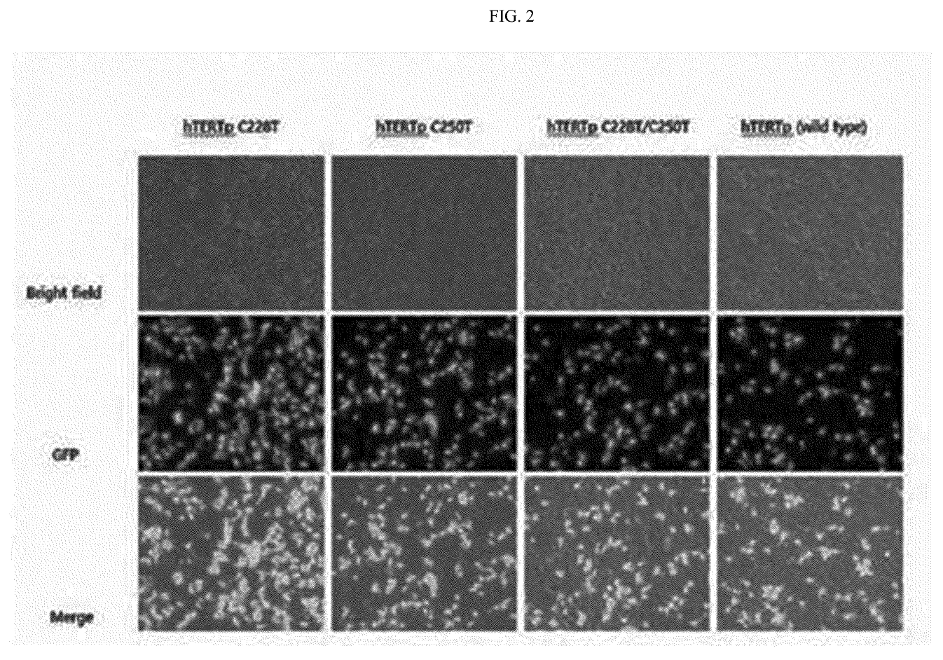

As a result, it was confirmed that when an adenovirus was prepared using an hTERT promoter (C228T) of the present invention, a virus with 2.28 times higher infectivity than that of a wild-type may be produced (see FIG. 2 and Table 1).

TABLE 1

| | | | Production/ | Fold |
|---|---|---|---|---|
| | Input | Output | cell | increase |
| hTERTp C228T | 5 × 10\hat{ }4 Ifu | 4.8 × 10\hat{ }8 Ifu | 900 Ifu | 2.28 |
| hTERTp C250T | 5 × 10\hat{ }4 Ifu | 2.0 × 10\hat{ }8 Ifu | 400 Ifu | 0.95 |
| hTERTp | 5 × 10\hat{ }4 Ifu | 2.4 × 10\hat{ }8 Ifu | 480 Ifu | 2.14 |
| C228T/C250T | | | | |
| hTERTp | 5 × 10\hat{ }4 Ifu | 2.1 × 10\hat{ }8 Ifu | 420 Ifu | 1 |
| (wild type) | | | | |

### [Example 3] Confirmation of E4 Expression Ability when hTERT Promoter of the Present Invention was Introduced into Adenovirus

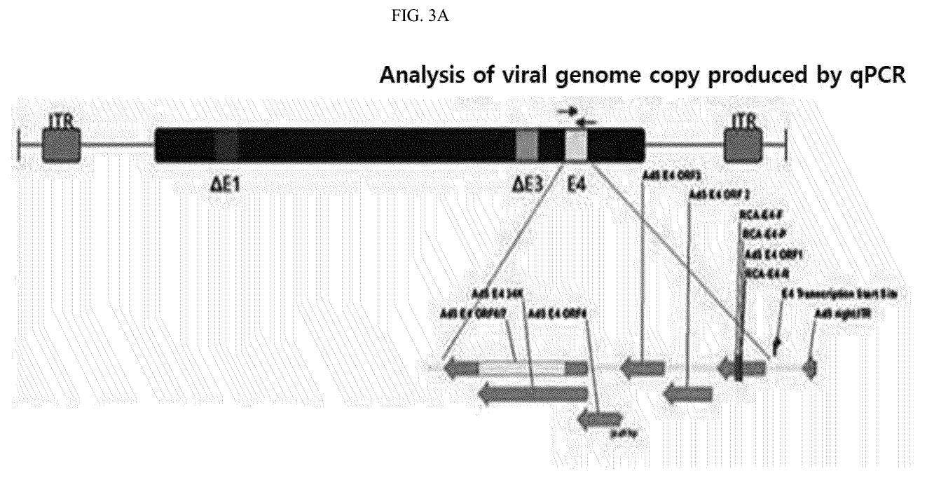

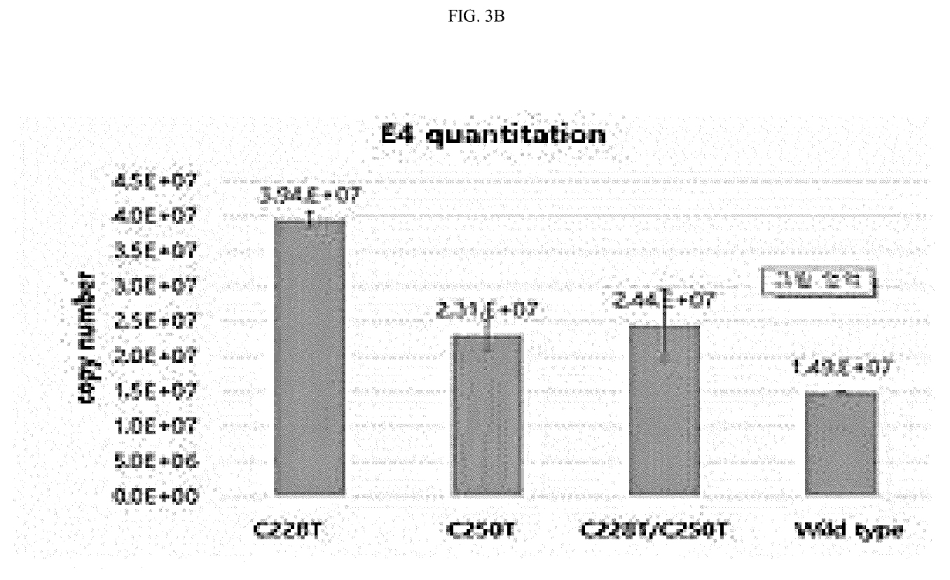

2×10⁶ cells of HEK-293 cells were inoculated in a 6-well plate, and then the cells included in each well were infected in 0.1 MOI. After 48 hours, the cells included in each well and the cells in a virus culture medium were lyzed to recover the produced virus. gDNA was extracted from the recovered virus and the expression of E4 was quantified by qPCR (FIG. 3A). The E4 primers used in the experiment were as follows. E4_F: 5′-GAC TCG GTA AAC ACA TCA GGT TGA-3′, E4_R: 5′-GGG CTA TTT CGG TCG CTT TT-3′

As illustrated in FIG. 3B, it was confirmed that when an adenovirus was prepared using a C228T mutant hTERT promoter, the E4 expression ability was remarkably increased compared to a case where an adenovirus was prepared using a wild-type hTERT promoter.

### [Example 4] Confirmation of Tumor Killing Ability when hTERT Promoter of the Present Invention was Introduced into Adenovirus

With respect to an adenovirus to which hTERT promoters C228T, C250T, and C228T/C250T of the present invention were introduced into a bladder cancer cell line 253J, the apoptotic effect was confirmed.

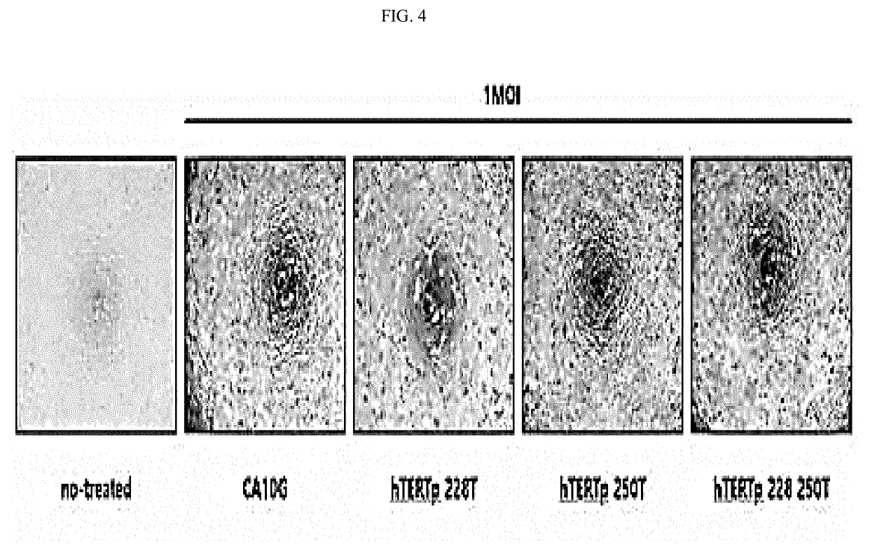

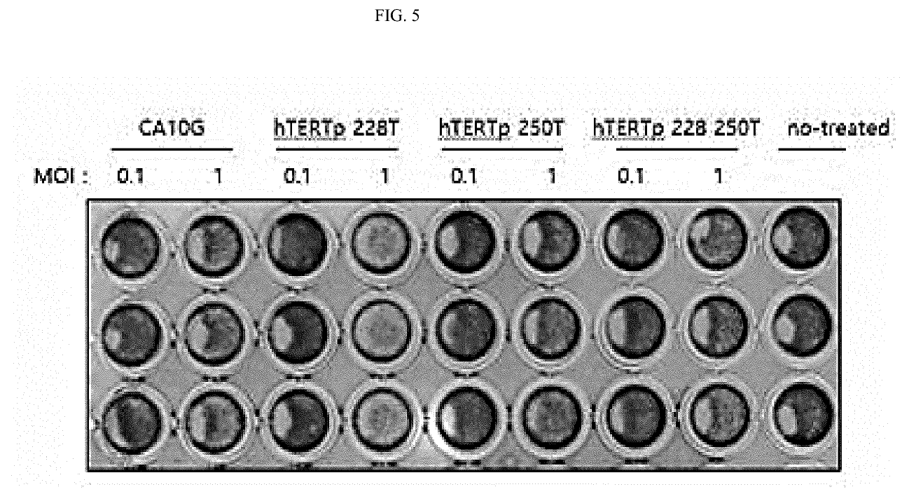

More specifically, the bladder cancer cell line 253J was inoculated in a 96-well plate at 5×10³ cells/well, and then the virus was treated for each MOI. After 72 hours, the culture medium was fully removed and 30 uL per well was treated with a crystal violet solution (crystal violet 0.5%, acetic acid 0.5%, methanol 95%). After incubation (fixation & staining) for 20 minutes at room temperature, the crystal violet solution was removed. After being slowly washed in running water, the plate was air-dried to dry completely. Data were secured by photographing the plate with Gell-doc and measuring the stained area with image J. The results thereof were shown in FIGS. 4 and 5.

As illustrated in FIGS. 4 and 5, it was confirmed that the tumor killing ability of the adenovirus introduced with the hTERT promoter of the present invention was significantly higher than that of the adenovirus introduced with the wild-type hTERT promoter.

### [Example 5] Confirmation of E1A Expression Ability when hTERT Promoter of the Present Invention was Introduced into Adenovirus

2×10⁶ cells of 253J cells were inoculated in a 6-well plate, and then the cells included in each well were infected in 1 MOI. After 48 hours, the cells included in each well and the cells in a virus culture medium were lyzed to recover the produced virus. gDNA was extracted from the recovered virus, and the expression of E1A was confirmed by PCR. The primers used to confirm the expression of E1A were as follows. E1A_F: 5′-tcc ggt cct tct aac aca cc-3′, E1A_R: 5′-ggc gtt tac agc tca agt cc-3′, GAPDH_F: 5′-tgt gca cca cca act get tag c-3′, GAPDH_R: 5′-ggc atg gac tgt ggt cat gag-3′

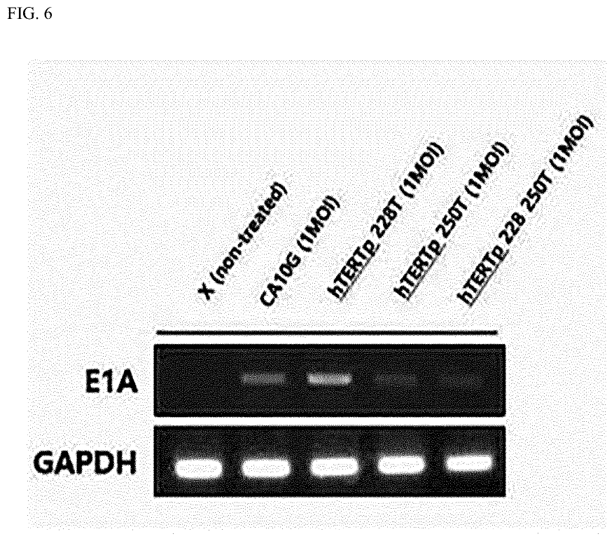

As illustrated in FIG. 6, it was confirmed that the expression level of E1A of the adenovirus introduced with the hTERT promoter of the present invention was significantly increased compared to that of the adenovirus introduced with the wild-type hTERT promoter.

### [Preparation Example 1] Adenovirus Including hTERT Variant Promoter

The endogenous gene of the adenovirus of the present invention has a structure of 5′ITR-C1-C2-C3-C4-C5 3′ITR; in which the C1 includes E1A, E1B or E1A-E1B. The C2 includes E2B-L1-L2-L3-E2A-L4. The C3 does not include E3 or includes E3; the C4 includes L5; and the C5 includes E4, and a cancer-specific adenovirus was prepared by inserting the hTERT variant promoter of the present invention into the E1 region.

### [Preparation Example 2] Adenovirus Expressing Dual-Target shRNA Including hTERT Variant Promoter

An adenovirus expressing dual-target shRNA was intended to be prepared using the adenovirus including the variant promoter prepared in Preparation Example 1 above. The dual-target shRNA of the present invention is included in the E3 region of C3 in the endogenous gene structure of the adenovirus described in Preparation Example 1 above. Specifically, the dual-target shRNAs of the present invention may include: 1) shRNA that dual-targets mTOR/STAT3; 2) shRNA that dual-targets AR/mTOR; 3) shRNA that dual-targets BCL2/B1-1; and 4) shRNA that dual-targets c-MET/PD-L1. The 1) shRNA that dual-targets mTOR/STAT3 is described in detail in Korean (KR) Patent Registration No. 10⁻¹⁸⁶⁵⁰²⁵ and Korean (KR) Patent Registration No. 10⁻²⁰³⁴⁷⁶⁴, and the 2) shRNA that dual-targets AR/mTOR is described in detail in Korean (KR) Patent Registration No. 10-1999515, Korean (KR) Patent Registration No. 10⁻²¹⁴⁵⁶⁶⁴ and Korean (KR) Patent Publication No. 10⁻²⁰²¹-0118760. The 3) shRNA that dual-targets BCL2/B1-1 is described in detail in Korean (KR) Patent Registration No. 10⁻¹⁹⁹³³⁷⁷ and Korean (KR) Patent Registration No. 10⁻²¹⁴⁵⁶⁶⁵, and the 4) shRNA that dual-targets c-MET/PD-L1 is described in detail in Korean (KR) Patent Registration No. 10⁻²⁰²¹-0032716. The dual-target shRNA was included in the E3 region of C3 in the endogenous gene structure of the adenovirus to construct a dual-target shRNA expressing adenovirus including the hTERT variant promoter.

1. A promoter in which a 228th nucleotide sequence of an hTERT promoter is substituted from C to T; and/or a 250th nucleotide sequence is substituted from C to T.

2. The promoter of claim 1, further comprising:
a substitution selected from the group consisting of a substitution from T to C in a 129th nucleotide sequence; a substitution from C to T in a 235th nucleotide sequence; a substitution from C to T of a 236th nucleotide sequence; a substitution from C to T of a 249th nucleotide sequence; and/or a substitution from A to C of a 317th nucleotide sequence of the hTERT promoter.

3. The promoter of claim 1, further comprising:
a deletion of a 245th nucleotide sequence of the hTERT promoter.

4. The promoter of claim 1, wherein the promoter overexpresses a target gene in a cancer cell-specific manner.

5. The promoter of claim 1, wherein the promoter is a promoter represented by SEQ ID NO: 2.

6. The promoter of claim 1, wherein the promoter is a promoter represented by SEQ ID NO: 3.

7. The promoter of claim 1, wherein the promoter is a promoter represented by SEQ ID NO: 4.

8. A composition for overexpressing a target gene in a cancer cell-specific manner comprising:
a cancer cell-specific promoter in which a 228th nucleotide sequence of an hTERT promoter is substituted from C to T; and/or a 250th nucleotide sequence is substituted from C to T; and
a target gene operably linked to the promoter.

9. The composition of claim 8, further comprising:
a substitution selected from the group consisting of a substitution from T to C in a 129th nucleotide sequence; a substitution from C to T in a 235th nucleotide sequence; a substitution from C to T of a 236th nucleotide sequence; a substitution from C to T of a 249th nucleotide sequence; and/or a substitution from A to C of a 317th nucleotide sequence of the hTERT promoter.

10. The composition of claim 8, wherein the promoter further includes a deletion of a 245th nucleotide sequence of the hTERT promoter.

11. The composition of claim 8, wherein the target gene is at least one gene selected from the group consisting of a cancer cell death gene, a fluorescent protein gene, a cancer cell suppressor gene, an antigenic gene, a cytotoxic gene, a cell proliferation inhibitory gene, a cytokine gene, a pro-apoptotic gene, and an anti-angiogenic gene.

12. The composition of claim 11, wherein the cancer cell death gene is a BCL-2 family pro-apoptotic gene or a death receptor/ligand gene.

13. The composition of claim 11, wherein the fluorescent protein gene is at least one selected from the group consisting of luciferase, an enhanced green fluorescent protein (EGFP), a green fluorescent protein (GFP), a yellow fluorescent protein (YFP), a red fluorescent protein (RFP), and a cyan fluorescent protein (CFP).

14. The composition of claim 11, wherein the cancer cell suppressor gene is at least one selected from the group consisting of a p53 gene, an APC gene, a DPC-4/Smad4 gene, a BRCA-1 gene, a BRCA-2 gene, a WT-1 gene, an MMAC-1 gene, an MMSC-2 gene, an NF-1 gene, an MTS1 gene, a CDK4 gene, an NF-1 gene, an NF-2 gene, and a VHL gene.

15. A recombinant expression vector comprising the promoter of claim 1.

16. A transformant transformed with the recombinant expression vector of claim 15.

17. An oncolytic adenovirus comprising a cancer cell-specific promoter in which a 228th nucleotide sequence of an hTERT promoter is substituted from C to T; and/or a 250th nucleotide sequence is substituted from C to T.

18. The oncolytic adenovirus of claim 17, wherein the promoter further includes:
a substitution selected from the group consisting of a substitution from T to C in a 129th nucleotide sequence; a substitution from C to T in a 235th nucleotide sequence; a substitution from C to T of a 236th nucleotide sequence; a substitution from C to T of a 249th nucleotide sequence; and/or a substitution from A to C of a 317th nucleotide sequence of the hTERT promoter.

19. The oncolytic adenovirus of claim 17, wherein the promoter further includes a deletion of a 245th nucleotide sequence of the hTERT promoter.

20. The oncolytic adenovirus of claim 17, wherein the adenovirus has increased tumor killing ability compared to an adenovirus including a wild-type hTERT promoter.

21. The oncolytic adenovirus of claim 20, wherein the adenovirus has increased E1A expression compared to the adenovirus including the wild-type hTERT promoter.

22. The oncolytic adenovirus of claim 17, wherein the promoter is operably linked to E1A and E1B.

23. A method for treating cancer, comprising administering a pharmaceutically effective dose of the oncolytic adenovirus of claim 17 to a subject.


SEQUENCE LISTING (4 sequences)

SEQ ID NO: 1 - DNA, 455 bp, Artificial Sequence
  wt-hTERT promoter
tggcccctccctcgggttaccccacagcctaggccgattcgacctctctccgctggggccctcgctggcgtccctgcaccctgggagcgcgagcggcgcgcgggcggggaagcgcggcccagacccccgggtccgcccggagcagctgcgctgtcggggccaggccgggctcccagtggattcgcgggcacagacgcccaggaccgcgctccccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgcg

SEQ ID NO: 2 - DNA, 455 bp, Artificial Sequence
  C250T hTERT promoter
tggcccctccctcgggttaccccacagcctaggccgattcgacctctctccgctggggccctcgctggcgtccctgcaccctgggagcgcgagcggcgcgcgggcggggaagcgcggcccagacccccgggtccgcccggagcagctgcgctgtcggggccaggccgggctcccagtggattcgcgggcacagacgcccaggaccgcgctccccacgtggcggagggactggggacccgggcacccgtcctgccccttcaccttccagctccgcctcctccgcgcggaccccgccccgtcccgaccccttccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgcg

SEQ ID NO: 3 - DNA, 455 bp, Artificial Sequence
  C228T hTERT promoter
tggcccctccctcgggttaccccacagcctaggccgattcgacctctctccgctggggccctcgctggcgtccctgcaccctgggagcgcgagcggcgcgcgggcggggaagcgcggcccagacccccgggtccgcccggagcagctgcgctgtcggggccaggccgggctcccagtggattcgcgggcacagacgcccaggaccgcgctccccacgtggcggagggactggggacccgggcacccgtcctgccccttcaccttccagctccgcctcctccgcgcggaccccgccccgtcccgacccctcccgggtccccggcccagccccttccgggccctcccagcccctccccttcctttccgcggccccgccctctcctcgcggcgcgagtttcaggcagcgctgcgtcctgctgcgcacgtgggaagccctggccccggccacccccgcg

SEQ ID NO: 4 - DNA, 455 bp, Artificial Sequence
  C250T/C288T-hTERT promoter
tggcccctccctcgggttaccccacagcctaggccgattcgacctctctccgctggggccctcgctggcgtccctgcaccctgggagcgcgagcggcgcgcgggcggggaagcgcggcccagacccccgggtccgcccggagcagctgcgctgtcggggccaggccgggctcccagtggattcgcgggcacagacgcccaggaccgcgctccccacgtggcggagggactggggacccgggcacccgtcctgccccttcaccttccagctccgcctcctccgcgcggaccccgccccgtcccgaccccttccgggtccccggcccagccccttccgggccctcccagcccctccccttcctttccgcggccccgccctctcctcgcggcgcgagtttcaggcagcgctgcgtcctgctgcgcacgtgggaagccctggccccggccacccccgcg